(19) United States
(12) Patent Application Publication
(10) Pub. No.: US 2023/0277043 A1
(43) Pub. Date: Sep. 7, 2023

(54) APPARATUS AND METHODS FOR LUBRICATING DEVICES INTRODUCED INTO A BODY OF A PATIENT

(71) Applicant: Dennis Lewis, Laramie, WY (US)

(72) Inventor: Dennis Lewis, Laramie, WY (US)

(21) Appl. No.: 18/106,202

(22) Filed: Feb. 6, 2023

Related U.S. Application Data
(63) Continuation-in-part of application No. 16/503,384, filed on Jul. 3, 2019, now Pat. No. 11,571,117.
(60) Provisional application No. 62/693,974, filed on Jul. 4, 2018.

(51) Int. Cl. A61B 1/00; A61B 1/31

(52) U.S. Cl. CPC A61B 1/00131; A61B 1/31

(57) ABSTRACT

Apparatus and methods are provided for lubricating a medical device, such as a colonoscope, being introduced into a patient's body. In one example, the apparatus includes a housing including a proximal surface, a distal surface, an interior communicating with the proximal and distal surfaces; and a lubricant within the interior of the housing such that a scope or other device inserted through the interior receives lubricant on its outer surface before introduction into a patient's body.

### RELATED APPLICATION DATA

The present application is a continuation-in-part of co-pending U.S. application Ser. No. 16/503,384, filed Jul. 3, 2019, issuing as U.S. Pat. No. 11,571,117, which claims benefit of U.S. provisional application Ser. No. 62/693,974, filed Jul. 4, 2018, the entire disclosures of which are expressly incorporated by reference herein.

### TECHNICAL FIELD

The present application relates to apparatus and methods for facilitating introduction of devices into a patient's body, e.g., for automatically lubricating colonoscopes, endoscopes, or other elongate devices during introduction through the rectum into the colon or other natural orifice and/or body lumens.

### BACKGROUND

During endoscopic procedures, a colonoscope, endoscope, or other device may be inserted through the rectum into a patient's colon, e.g., to acquire images and/or perform a medical procedure within the patient's gastrointestinal tract. To facilitate insertion, lubricant, e.g., water-based jelly, petroleum jelly, or other lubricant, may be applied to the distal end of the device and/or to the patient's body around the rectum. However, such manual application may be messy and/or result in uneven application of the lubricant and may require the user to then wash and/or otherwise clean their hands to allow subsequent manipulation of the endoscope and completion of the procedure.

Accordingly, apparatus and methods for facilitating application of lubricant would be useful.

### SUMMARY

The present application is directed to apparatus and methods for facilitating introduction of devices into a patient's body, e.g., for automatically lubricating colonoscopes, endoscopes, and/or other elongate devices during introduction through the rectum into the colon or other natural orifice and/or body lumens.

In accordance with one example, an apparatus is provided for lubricating a medical device being introduced into a patient's body that includes a housing including a proximal surface, a distal surface, a channel extending between the proximal and distal surfaces; and a lubricant within an interior of the housing surrounding the channel such that a device inserted through the channel receives lubricant on its outer surface before introduction into a patient's body.

In accordance with another example, a method is provided for introducing a medical device into a body passage of a patient's body that includes providing a lubricant apparatus including lubricant within an interior surrounding a channel through the apparatus; introducing a distal end of a medical device into and through the channel, whereupon lubricant from the interior is applied to an outer surface of the medical device; and introducing the distal end of the medical device into the body passage to perform a procedure, lubricant being applied to the outer surface of the medical device as the medical device passes through the channel and into the body passage.

In accordance with still another example, a device is provided for lubricating a medical device being introduced into a patient's body that includes a housing comprising a proximal surface including a proximal opening, a distal surface including a distal opening, and an interior within the housing communicating with the proximal and distal openings; adhesive or tacky material on the distal surface at least partially surrounding the distal opening to secure the distal surface to the patient around the rectum; and a lubricant within the interior of the housing such that a device inserted through the housing between the proximal and distal openings receives lubricant on its outer surface before introduction into a patient's body.

In accordance with still another example, a device is provided for lubricating a medical device being introduced into a patient's body that includes a housing comprising a proximal housing portion including a proximal surface including a proximal opening and a sidewall extending from the proximal surface to define a cavity; a distal housing portion comprising a distal surface including a distal opening, the distal housing portion attached to the proximal housing portion to define an interior within the housing communicating with the proximal and distal openings; and a flange defining a distal-most surface of the housing. Adhesive or tacky material may be provided on the distal surface to secure the distal surface to the patient around the rectum, and a lubricant may be provided within the interior of the housing such that a device inserted through the housing between the proximal and distal openings receives lubricant on its outer surface before introduction into a patient's body.

In accordance with another example, a method is provided for introducing a medical device into a body passage of a patient's body that includes providing a lubricant device including lubricant within an interior of a housing of the device communicating with proximal and distal openings; introducing a distal end of a medical device into and through the interior between the proximal and distal openings, whereupon lubricant from the interior is applied to an outer surface of the medical device; and introducing the distal end of the medical device into the body passage to perform a procedure, lubricant being applied to the outer surface of the medical device as the medical device passes through the interior and into the body passage.

Other aspects and features of the present invention will become apparent from consideration of the following description taken in conjunction with the accompanying drawings.

### BRIEF DESCRIPTION OF THE DRAWINGS

The invention is best understood from the following detailed description when read in conjunction with the accompanying drawings. It is emphasized that, according to common practice, the various features and design elements of the drawings are not to-scale. On the contrary, the dimensions of the various features and design elements are arbitrarily expanded or reduced for clarity. Included in the drawings are the following figures.

FIGS. 1 and 2 are end and cross-sectional side views, respectively, of an example of a lubricating apparatus for applying lubricant to a medical device inserted through a channel in the apparatus.

FIG. 3 shows a perspective view and a side view of a ring that may be provided around the channel of the apparatus of FIGS. 1 and 2 to automatically deliver lubricant to the outer surface of a device inserted through the channel.

FIGS. 4 and 5 show a colonoscope being inserted through the ring of FIG. 3.

FIGS. 6 and 7 are end and side views, respectively, of another example of a lubricating apparatus.

FIGS. 8A and 8B are perspective views of another example of a lubricating device for applying lubricant to a medical device inserted through a channel in the apparatus.

FIG. 8C is a partial cross-sectional view of the lubricating device of FIGS. 8A and 8B showing the interior of the device.

FIG. 9 is an exploded view of the lubricating device of FIGS. 8A-8C showing various exemplary components of the device.

FIGS. 10A and 10B are perspective and cross-sectional views of an exemplary bottom or distal housing portion for the lubricating device of FIGS. 8A-8C.

FIGS. 11A and 11B are perspective and cross-sectional views of an exemplary top or proximal housing portion for the lubricating device of FIGS. 8A-8C.

### DETAILED DESCRIPTION

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

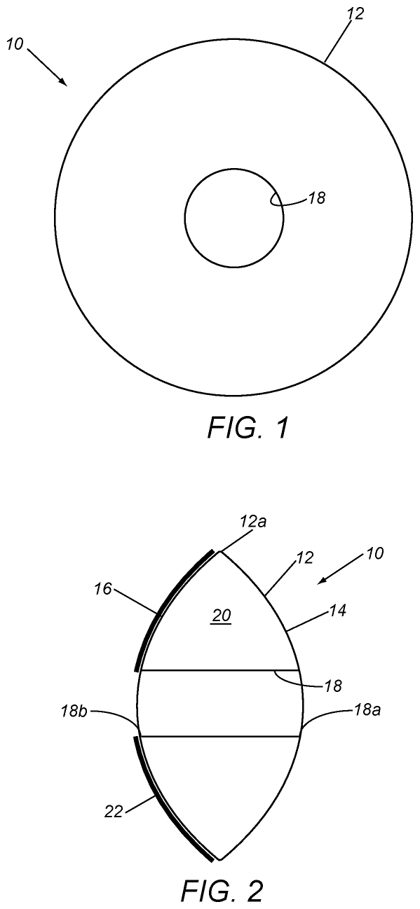

Turning to the drawings, FIGS. 1 and 2 show an example of a lubricating apparatus 10 or “jelly donut” device that includes an outer housing 12 including proximal and distal surfaces 14, 16, and a channel 18 extending between the surfaces 14, 16, e.g., from openings 18a, 18b. The housing 12 defines an inner annular chamber 20 surrounding the channel 18 that may be filled with lubricant, e.g., water-based lubricant, such as K-Y jelly, petroleum jelly, and the like.

As shown, the housing 12 may define an annular or “donut” shape, e.g., with the surfaces 14, 16 tapering from the channel 18 to an outer edge 12a defining an outer circumference for the housing 12. Optionally, the housing 12 may have other desired shapes, e.g., a distal surface 16 shaped to conforms to the patient's skin or other anatomy against which the apparatus 10 may be positioned (not shown), e.g., having a substantially flat, convex, concave, or other shape that may conform to the patient's anatomy. As shown, the surfaces 14, 16 may taper outwardly such they meet at the outer edge 12a, although alternatively, the surfaces may define a rounded outer edge and/or other shape (not shown).

In addition, the distal surface 16 may include an adhesive or other tacky material 22 that may cover all or a portion of the distal surface 16, e.g., at least around the outer edge 12a, immediately around the distal channel opening 18b, and the like, to facilitate anchoring and/or stabilizing the apparatus 10 against a patient's body, e.g., over the rectum (not shown). For example, the material 22 may be sufficiently tacky to secure the distal surface 16 to the patient's skin during a procedure, yet be removable upon completion of the procedure while leaving little or no residue on the patient's skin.

In one example, the housing 12 may be formed from a flexible and/or elastic material, e.g., plastic material having sufficient strength to contain the lubricant within the interior 20 yet being sufficiently flexible, e.g., to minimize risk of damage and/or to conform partially to the patient's skin or other contacted body. For example, the proximal surface 14 and/or distal surface 16 may include a flexible elastic cover with a hole or opening 18a, 18b in the center. One or both of the surfaces 14, 16 may be biased to a predetermined shape, yet the material may be flexible to conform to the patient's anatomy, e.g., such that the distal surface 16 may be deflected or deformed to be seated against the patient's skin.

Before use, e.g., when packaged and/or manufactured, the opening(s) 18a, 18b may be covered, e.g., with a removable sheet, cover, or other sealing member (not shown) to prevent the lubricant within the interior 20 from escaping from the apparatus 10. Alternatively, an annular cover (also not shown) may be provided that extends along the channel 18 to prevent lubricant from leaking from the chamber 20. The cover may be slid out of the channel 18 or may be peeled, torn, or otherwise removed before use to expose the channel 18 to the lubricant.

Optionally, the proximal opening 18a may include an annular taper, ramp, bell-mouth and/or the like (not shown) to facilitate guiding a device being inserted into the channel 18.

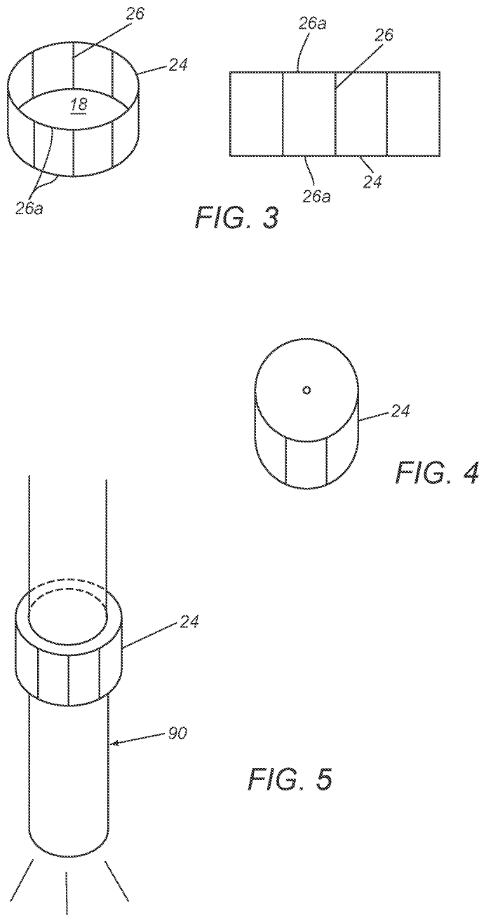

In the example shown in FIGS. 3-5, the channel 18 may be defined by a ring 24 extending between the proximal and distal openings 18a, 18b in the proximal and distal surfaces 14, 16. The ring 24 may include one or more openings, e.g., between a plurality of ribs or other scaffold structure, to allow lubricant within the interior 20 to enter the channel 18. For example, as shown in FIG. 3, the ring 24 may include a plurality of struts or longitudinal elements 26 that extend longitudinally along the channel 18, e.g., between the proximal and distal surfaces (not shown in FIG. 3). Optionally, the ring 24 may include one or more annular struts, webbing, and/or other structures, e.g., two annular elements 26a shown in FIG. 3, to define a channel 18 of the apparatus 10 yet provide openings for the lubricant within the interior 20 to enter the channel 18.

In one example, the ring 24 may be sufficiently rigid to define a substantially fixed diameter, e.g., having a diameter slightly larger than the diameter of an colonoscope or other device 90 (shown in FIG. 5) that may be introduced through the channel 18, e.g., greater than 10-13 mm. Alternatively, the ring 24 may be formed from elastic material to allow the ring 24 to expand to accommodate different size devices being inserted through the channel 18 yet providing a close fit, e.g., to provide a partial seal around the device 90 to prevent leakage of the lubricant, particularly through the opening 18a in the proximal surface 14.

In another example, the ring 24 may be replaced by an annular wall that includes a plurality of openings or pores having cross-sections sufficiently large to allow lubricant to pass through the wall into the channel 18 at a desired rate, while preventing excessive lubricant from escaping. Optionally, the openings may include valve elements, e.g., individual flaps formed in the wall or attached over the openings (not shown), which may open when a device is directed though the channel 18 to deliver lubricant, yet may close when nothing is in the channel 18 to prevent the lubricant from leaking. In another option, the wall may be formed from permeable material that has pores sufficiently large to allow the lubricant to pass through the wall into the channel 18.

The openings and/or pores may be evenly distributed around the perimeter and/or length of the wall. Alternatively, the openings or pores may be provided only along a portion of the channel 18, e.g., from the proximal surface 14 only partially along the length of the channel 18 towards the distal surface 16.

When ready to use, any cover or sealing member may be removed to uncover the opening(s) 18a, 18b and/or the distal surface 16, and a distal end of a colonoscope or other device 90 may be inserted through the proximal opening 18a in the proximal surface 14 (i.e., the endoscopist or user side) into the channel 18, which, optionally, may stretch to facilitate passage of the scope 90 into the channel 18. Optionally, the ring 24 and/or housing 18 may be formed from elastic material that provides a close elastic fit around the scope 90, e.g., to remove excess lubricant when the scope 90 exits the channel 18 through the distal opening 18b.

If the distal surface 16 (i.e., patient side of the channel 18) includes a removable, e.g., annular, cover (not shown) over the adhesive 22, the cover may be removed before or after inserting the scope 90 through the channel. The distal opening 18b may be slightly larger than the scope 90 (e.g., greater than 10-13 mm diameter) to allow the lubricant to stay on the outer surface of the scope 90 and/or provide a squeegee to apply a desired thickness of lubricant on the outer surface as the scope 90 passes into the patient, e.g., into the rectum.

After initial insertion through the channel 18, the scope 90 may be introduced into the patient's body, e.g., through the rectum into the colon (not shown). The apparatus 10 may then be seated against the patient, e.g., thereby pressing the distal surface 16 against the patient's skin surrounding the rectum, which may apply the adhesive to the skin to prevent the apparatus 10 from moving during manipulation of the scope 90. Alternatively, the distal surface 16 of the apparatus 10 may be placed against the patient's skin, e.g., with the channel 18 aligned with the rectum, before introducing the scope 90.

With the apparatus 10 shown in FIGS. 1 and 2, as the scope 90 is advanced, lubricant may be automatically applied to the outer surface of the scope 90 as it passes through the channel 10, thereby facilitating advancement of the scope 90 into the patient's colon. The scope 90 may then be used to perform a procedure, e.g., observing and/or treating the patient's colon. Upon completing the procedure, the scope 90 may be removed, and then the apparatus 10 may be removed. Optionally, one more additional instruments may be introduced through the channel 18 before removing the apparatus 10.

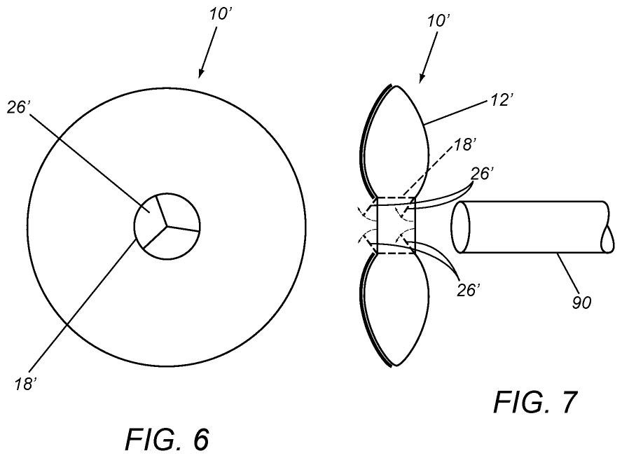

Alternatively, as shown in FIGS. 6 and 7, a lubricating apparatus 10′ may be provided that is constructed generally similar to the previous example, e.g., including a housing 12′ having a channel 18′ therethrough. In this alternative, one-way valves 26 may be provided that surround and/or seal the proximal and distal openings 18a′, 18b′ communicating with the channel 18′. The valves 26 may be biased to closed positions but may be movable to open positions to allow insertion of a scope or other device 90 into and through the channel 18′ with the scope 90 receiving lubricant within the housing 12′ on its outer surface as it passes through the channel 18′, similar to the previous examples. Upon completion of the procedure, the scope 90 may be removed from the colon through the channel 18,′ whereupon the valves 26 may automatically close as the apparatus 10 is removed.

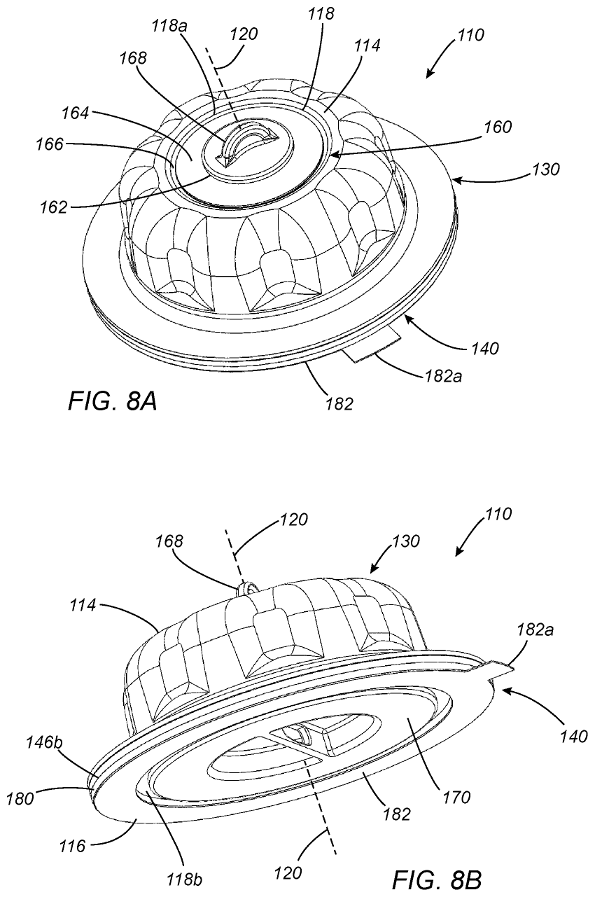

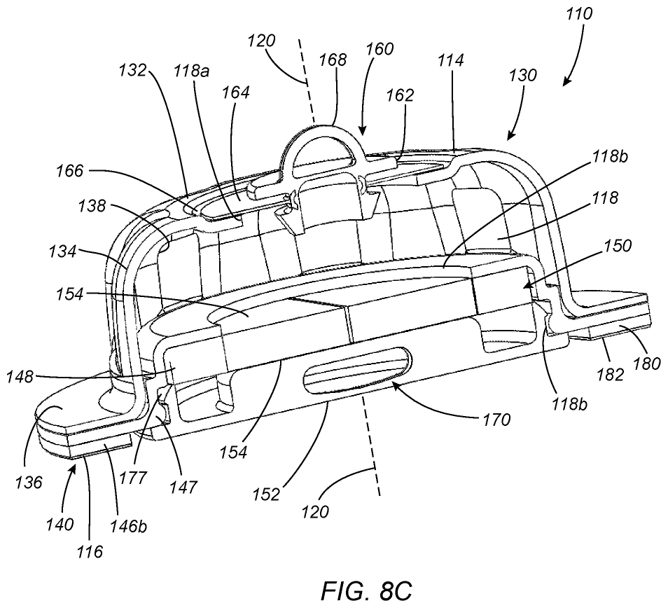

Turning to FIGS. 8A-8C, another example of a lubricating device 110 is shown that includes an outer housing 112 including proximal and distal ends or surfaces 114, 116, and a channel, passage, or other interior region 118 extending at least partially between the surfaces 114, 116 that may contain a lubricant (not shown). For example, the proximal and distal surfaces 114, 116 may include proximal and distal openings 118a, 118b, respectively, that may communicate with the interior 118 such that a device introduced between the openings 118a, 118b through the interior 118, e.g., along axis 120, may have lubricant applied before insertion into a patient's body, generally similar to the other devices described elsewhere herein.

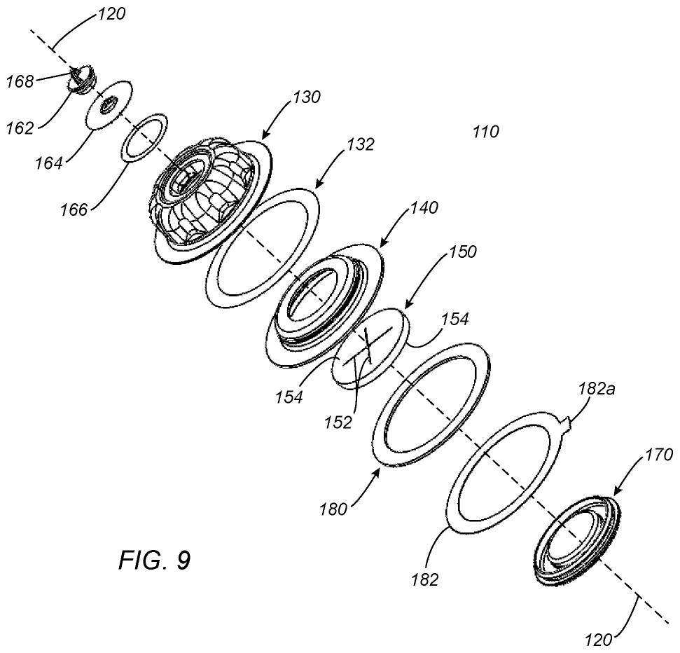

With additional reference to FIG. 9, in this example, the housing 112 includes a top or proximal housing portion 130 and a bottom or distal housing portion 140 that may be fabricated separately and attached together to enclose the interior 118 and the lubricant therein. For example, each of the housing portions 130, 140 may be made using conventional materials and methods, e.g., formed from plastic, metal, or composite materials, by one or more of molding, casting, machining, and the like. The housing portions 130, 140 may then be permanently attached together, e.g. using one or more of an adhesive seal 132, cooperating connectors (not shown), fusing, sonic welding, and the like.

Optionally, the device 110 may include one or more additional components, e.g., one or more valves, seals, covers, and the like to selectively close the openings 118a, 118b and/or minimize the lubricant escaping from the interior 118 during use. For example, as best seen in FIG. 8C, a slit valve 150 may be mounted within the housing 112, e.g., adjacent the distal opening 118b, that may be biased closed but may open to accommodate inserting a medical device through the interior 118 during use of the device 110. The valve 150 may be formed from flexible material, e.g., jelly foam, and/or plastic material, that may be sufficiently flexible to allow a medical device to be inserted through the valve 150 while providing a seal around the medical device.

In the example shown, the slit valve 150 includes a pair of intersecting slits 152 extending between opposite surfaces 154 of the valve 150. The slits 152 may be cut or otherwise formed through the body of the valve 150, e.g., using one or more of blades, dies, a laser, and the like (not shown), e.g., using conventional methods. For example, the slits 152 may allow the adjacent portions of the valve 150 to open when a medical device is inserted through the valve 150 to provide a seal around the medical device, with the valve 150 resiliently closing when the medical device is removed to close the slits 152 and prevent substantial leakage of lubricant from the device 110 and/or bodily fluids adjacent the device 110 entering the interior 118.

In addition, the device 110 may include one or more covers or seals, e.g., for closing the openings 118a, 118b before use. For example, a first seal member 160 may be provided that is sized to close the first opening 118a, e.g., which may be selectively removed before use. In the example shown, the first seal member 160 includes a plug member 162 attached to a circular seal 164, which may be configured to removably engage a corresponding seal ring 166, which may be attached to the proximal opening 118a.

For example, the plug 162 and seal 164 may be formed separately and permanently attached together, e.g., by one or more of cooperating connectors, bonding with adhesive, fusing, sonic welding, and the like, or, alternatively, may be integrally formed together as a single piece. The seal ring 166 may be attached to the proximal housing portion 130, e.g., by one or more of cooperating connectors, bonding with adhesive, fusing, sonic welding, and the like. For example, the seal ring 166 may be formed from material that is more flexible than the proximal housing portion 130, e.g., to accommodate removing the seal member 160 to access the opening 118a, and, optionally, reclosing the opening 118a with the seal member 160, if desired.

Similarly, a cap 170 may be provided for closing the distal opening 118b before use. For example, as best seen in FIG. 8C, the distal housing portion 140 may include a recess 148 adjacent the distal opening 118b sized to receive the cap 170, thereby closing the distal opening 118b, as described further herein.

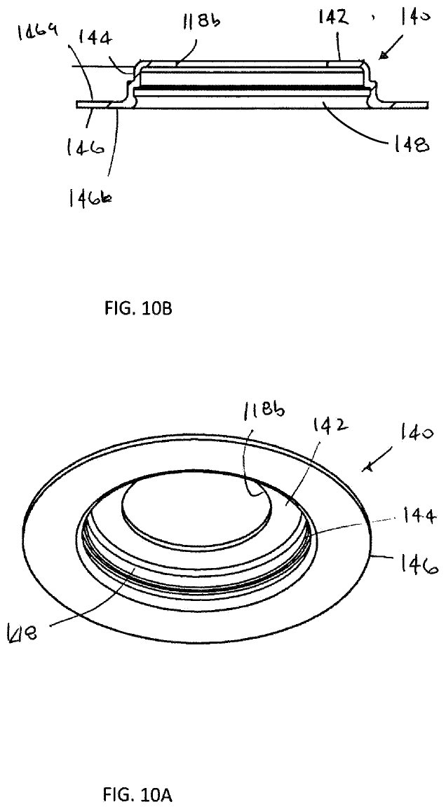

With additional reference to FIGS. 10A and 10B, the distal housing portion 140 may include an upper surface 142 including the distal opening 118b therein, an annular sidewall 144 extending distally from the upper surface 142, and a flange 146 extending radially outwardly from the sidewall 144, e.g., thereby defining recess 148 therein. The flange 146 may include proximal and distal surfaces 146a, 146b opposite one another, e.g., such that the flange 146 defines a substantially planar distal surface 146b for placement against a patient's body. Optionally, the flange 146 may have other shapes if desired, e.g., to provide a curved distal surface (not shown), e.g., similar to other devices herein.

The distal housing portion 140 may be formed from substantially rigid or semi-rigid material, e.g., plastic, metal, composite materials, and the like, by one or more of molding casting, machining, and the like, such that the flange 146 maintains its shape during use of the device 110. Alternatively, at least the flange 146 may have sufficient flexibility such that the shape of the distal surface 146b may be modified, e.g., to conform to the shape of the patient's body when the distal surface 146b is placed against the patient's skin.

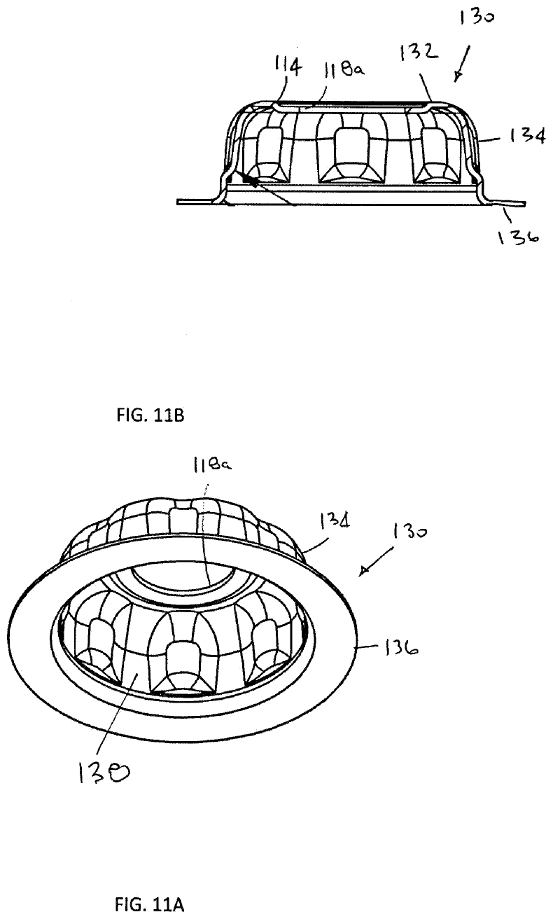

Turning to FIGS. 11A and 11B, an example of a proximal housing portion 130 is shown that may be attached to the lower housing portion 140, e.g., as shown in FIGS. 8A-8C and described further elsewhere herein. As shown, the proximal housing portion 130 includes an upper surface 132 including the proximal opening 118a, a sidewall 134 extending distally from the upper surface 132, and a flange 136 extending radially outwardly from the sidewall 134 opposite the upper surface 132. The sidewall 134 and upper surface 32 may define a rounded dome shape, as shown, or other desired shape, thereby providing a cavity 136 that may be enclosed to define the interior 118 of the device 110, as described further below.

Similar to the distal housing portion 140, the proximal housing portion 130 may be formed from substantially rigid or semi-rigid material, e.g., plastic, metal, composite materials, and the like, by one or more of molding casting, machining, and the like. The housing portions 130, 140 may be shaped such that the distal housing portion 140 may be partially received within the proximal housing portion 130 to enclose the interior 118 of the device.

For example, as best seen in FIG. 8C, the sidewall 144 of the distal housing portion 140 may be smaller than the sidewall 134 of the proximal housing portion 130 such that the distal housing portion 140 is partially received within the cavity 138 of the proximal housing portion 130. The flanges 136, 146 may limit insertion of the distal housing portion 140, e.g., such that the upper surfaces 132, 142 (and consequently the openings 118a, 118b) are spaced apart from one another when the flanges 136, 146 contact one another.

With additional reference to FIG. 8B, the lower surface 116 may include adhesive or tacky material, e.g., at least partially surrounding the distal opening 118b, which may be used to secure the device 110 to a patient during use. For example, a ring 180 of tacky material, e.g., hydrogel, adhesive material, and the like may be attached to the lower surface 146b of the flange 146 that may be used to secure the distal surface 116 to the patient, e.g., around the rectum (not shown), similar to other devices herein. Optionally, as shown, a seal or cover 182 may be provided that overlies the ring 180, which may be removed before use of the device 110, e.g., using a tab 182a or other feature to facilitate removal.

The device 110 may be manufactured and/or assembled and provided to a physician or other user with the interior 118 filled with lubricant and the seal member 160 and cap 170 enclosing the openings 118a, 118b. For example, during manufacturing, the housing portions 130, 140 may be formed and permanently attached together, e.g., by attaching the flanges 136, 146 together. The valve 150 may be inserted into the recess 148 of the distal housing portion 140 before or after assembling the housing portions 130, 140 together. The valve 150 may simply be inserted or otherwise seated within the recess 148 against the upper surface 142 or, optionally, the valve 150 may be permanently attached to the distal housing portion 140, e.g., using one or more of bonding with adhesive, fusing, sonic welding, cooperating connectors, and the like.

The cap 170 may be inserted into the recess 148 of the distal housing portion 140 any time after seating the valve 150 to close the distal opening 118b. The cap 170 and/or distal housing portion 140 may include one or more features to secure the cap 170, while allowing subsequent removal. For example, as shown in FIG. 8C, the cap 170 include an annular groove 177 and the flange 146 of the distal housing portion 140 may include a corresponding annular ring 147 that may be received in the groove 177 to secure the cap 170. The cap 170 and/or ring 147 may be sufficiently flexible to accommodate securing and removing the cap 170, as desired.

Finally, the seal member 160 may be assembled and secured to the proximal opening 118a, e.g., before or after assembling the other components of the device 110. Lubricant material may be introduced into the interior 118 of the housing 112 through one or both of the openings 118a, 118b, e.g., during manufacturing and/or assembly, e.g., before securing the seal member 160 and/or cap 170 to enclose the opening(s) used. Alternatively, the device 110 may be assembled without lubricant and, immediately before a procedure or at any later time, the seal member 160 and/or cap 170 may be removed to fill the interior 118 with a desired lubricant. This may extend storage of the device 110 before use, e.g., if the lubricant has a more limited shelf-life than the other materials of the device 110, and/or may allow a user to select one of a variety of available lubricants for introduction into the interior 118 before a procedure, if desired.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A device for lubricating a medical device being introduced into a patient's body, comprising:
a housing comprising a proximal surface including a proximal opening, a distal surface including a distal opening, and an interior within the housing communicating with the proximal and distal openings;
adhesive or tacky material on the distal surface at least partially surrounding the distal opening to secure the distal surface to the patient around the rectum; and
a lubricant within the interior of the housing such that a device inserted through the housing between the proximal and distal openings receives lubricant on its outer surface before introduction into a patient's body.

2. The device of claim 1, wherein the adhesive or tacky material surrounds the distal opening on the distal surface.

3. The device of claim 1, further comprising a valve member adjacent one of the proximal and distal openings, wherein the valve member is biased closed but may open to accommodate inserting the medical device through the interior.

4. The device of claim 3, wherein the valve member comprises a slit valve mounted within the housing adjacent the distal opening.

5. The device of claim 1, further comprising a removable cover overlying the adhesive or tacky material.

6. The device of claim 1, further comprising a removable seal covering one of the proximal and distal openings.

7. The device of claim 6, wherein the removable seal comprises a plug member configured to close the proximal opening.

8. The device of claim 6, wherein the removable seal comprise a cap configured to close the distal opening.

9. The device of claim 1, wherein the housing comprises a distal housing portion including a first surface including the distal opening therein, an annular sidewall extending from the first surface, and a flange extending from the sidewall opposite the first surface defining the distal surface.

10. The device of claim 9, wherein the first surface is spaced apart from the proximal surface such that the interior is located between the first surface and the proximal surface.

11. The device of claim 9, wherein the first surface is substantially flat.

12. The device of claim 9, wherein the first surface and sidewall define a recess, the flange surrounding an opening of the recess.

13. The device of claim 12, further comprising a valve member received within the recess adjacent the distal opening.

14. The device of claim 12, further comprising a cap removably received with the recess for preventing lubricant from escaping through the distal opening before the cap is removed.

15. The device of claim 9, wherein the housing further comprises a proximal housing portion comprising the proximal surface, a sidewall extending from the proximal surface to define a cavity, and a flange extending from the sidewall opposite the proximal surface.

16. The device of claim 15, wherein the sidewall of the proximal housing portion is sized such that the distal housing portion is partially received within the cavity.

17. The device of claim 16, wherein the flanges of the proximal and distal housing portions are attached together to secure the housing portions together.

18. The device of claim 17, wherein the proximal surface and the first surface are spaced apart from one another such that the interior is located between the proximal surface and the first surface.

19. A device for lubricating a medical device being introduced into a patient's body, comprising:
a housing comprising:
a proximal housing portion including a proximal surface including a proximal opening and a sidewall extending from the proximal surface to define a cavity;
a distal housing portion comprising a distal surface including a distal opening, the distal housing portion attached to the proximal housing portion to define an interior within the housing communicating with the proximal and distal openings; and
a flange defining a distal-most surface of the housing;
adhesive or tacky material on the distal surface to secure the distal surface to the patient around the rectum; and
a lubricant within the interior of the housing such that a device inserted through the housing between the proximal and distal openings receives lubricant on its outer surface before introduction into a patient's body.

20-40. (canceled)

41. A method for introducing a medical device into a body passage of a patient's body, comprising:
providing a lubricant device including lubricant within an interior of a housing of the device communicating with proximal and distal openings;
introducing a distal end of a medical device into and through the interior between the proximal and distal openings, whereupon lubricant from the interior is applied to an outer surface of the medical device; and
introducing the distal end of the medical device into the body passage to perform a procedure, lubricant being applied to the outer surface of the medical device as the medical device passes through the interior and into the body passage.

42-51. (canceled)
